(19) United States
(12) Patent Application Publication
(10) Pub. No.: US 2007/0239174 A1
(43) Pub. Date: Oct. 11, 2007

(54) Epilator with Glide Tweezers

(76) Inventor: Francis Yiu, Hong Kong (CN)

Correspondence Address: WHITE & CASE LLP;PATENT DEPARTMENT, 1155 AVENUE OF THE AMERICAS, NEW YORK, NY, 10036, US

(21) Appl. No.: 11/696,826

(22) Filed: Apr. 5, 2007

Related U.S. Application Data
(60) Provisional application No. 60/790,249, filed on Apr. 6, 2006.

(51) Int. Cl. A61B 17/50

(52) U.S. Cl. 606/133

(57) ABSTRACT

An epilator for plucking hair from a user's skin in a virtually pain-free manner is provided. The epilator comprises a motor located in a housing and a hair-plucking assembly. The hair-plucking assembly comprises at least one set of rotating discs and at least one set of rotating disc carriers. The diameter of the discs is different than the diameter of the disc carriers. The disc carriers are structurally configured to prevent a user's skin from being listed during epilation. During use, the discs may rotate from an open position to receive a hair on a user's skin, to a closed position when the hair is plucked.

This application claims the priority benefit of U.S. provisional patent application Ser. No. 60/790,249, filed on Apr. 6, 2006, the contents of which are incorporated herein in their entirety.

### FIELD OF THE INVENTION

The present invention is directed to the field of grooming products. More specifically, the invention is directed to an epilator for plucking hair from the skin in a virtually pain-free manner.

### BACKGROUND

Many individuals find it desirable to remove hairs from their body for various reasons. For example, women commonly remove hairs from their legs and underarms for aesthetic reasons, and men who engage in sports such as cycling or swimming will often remove body hairs to reduce resistance and increase speed.

Removal of body hair can be accomplished in a number of ways, including waxing, shaving, chemically depilating, and epilating. Waxing has drawbacks in that the process is generally painful and messy. Chemical depilatories use strong chemicals to dissolve hair and can result in chemical bums in certain individuals. Although shaving can be used to cut hairs, the hairs can regrow quickly, and care must be taken to prevent cuts and bleeding.

Epilators are devices which pluck hairs from the skin and are relatively inexpensive and easy to use, but can cause pain during use. Although users of epilators are generally advised to maintain the epilating head slightly above the skin, many users find it difficult to keep the head at the recommended height, and tend to apply the epilating head directly to the surface of the skin.

Although certain epilators use various techniques to purportedly reduce the amount of pain felt by a user, these epilators still cause a non-negligible amount of pain and discomfort during use. Therefore, there still remains an unmet need for an epilator which does not cause discomfort during use.

### BRIEF DESCRIPTION OF THE INVENTION

The present invention is directed to a new epilator for removing hair from the skin of a user. Advantageously, the inventive epilator eliminates or at least significantly reduces the sensation of pain which may be felt while a hair is plucked from the skin. The epilator is also relatively easy to manufacture.

The epilator comprises: a housing; a motor located in the housing; and a hair-plucking assembly for removal of hair from a user's skin. The hair-plucking assembly is mounted to the housing and is operationally connected to the motor. The hair-plucking assembly comprises at least one set of rotating discs and at least one set of rotating disc carriers, wherein the diameter of the discs is different than the diameter of the disc carriers. The discs rotate during use and thereby provide a tweezing action for removal of hair from the skin. During use, the discs may move or rotate from an open position to receive a hair on the user's skin, and a closed position when the hair is plucked.

Without being bound by theory, it is believed that a heightened sensation of pain results when the skin is raised during plucking or tweezing. In the present invention, the rotating disc carriers cause the skin to remain flat when hair is plucked, and thereby significantly reducing the sensation of pain felt during hair removal. In other words, the disc carriers are structurally configured to prevent a user's skin from being lifted while the discs are plucking hair from the skin. The disc carriers maintain the discs at a predetermined distance from a user's skin.

In this manner, the epilator significantly reduces or eliminates pain felt by a user. Consequently, the present invention provides for pain-free or virtually pain-free removal of body hairs. The epilator can be used on any portion of the body, such as the legs, arms, underarms, chest, and back.

The novel features of the new epilator can generally be used in combination with other features of known epilators, such as those having a shaver assembly, light assembly, or vibrational assembly. Examples of epilators are disclosed in U.S. Pat. No. 5,611,804, U.S. Pat. No. 6,261,301, and U.S. Pat. No. 6,740,097, assigned to Braun; U.S. Pat. No. 5,976,157 and U.S. Pat. No. 6,123,713, assigned to K.I.S. Ltd; and U.S. Pat. No. 6,689,143 and U.S. Pat. No. 6,613,057, assigned to Philips.

As previously stated, the diameter of the disc carriers is different than the diameter of the discs. In one embodiment, the diameter of the disc carriers is greater than the diameter of the discs. In an alternative embodiment, the diameter of the disc carriers is less than the diameter of the discs.

For example, the diameter of the disc carriers can be 0.1-3 mm greater or less than the diameter of the discs. In one embodiment, the disc carriers have a diameter which is 0.6 mm greater than the diameter of the discs.

There may be any number of sets of disc or disc carriers in the epilator. For example, there may be a single set of discs and/or disc carriers, or there may be a plurality of such discs and/or disc carriers. Regardless of the number of discs and/or disc carriers, the diameter of the discs is different than the diameter of the disc carriers.

A set of discs may comprise any number of separate discs. For example, a set of discs may comprise a pair of discs, or three or more discs.

The set of discs may optionally comprise two outer discs and an inner disc. In such an embodiment, the outer discs may have the same diameter, and the inner disc may have a different diameter, such as a smaller or larger diameter, compared to the outer discs.

The disc carriers may be conveniently located on opposite sides of the discs. In other words, a set of disc carriers may “sandwich” a set of discs so that the discs are located in between the disc carriers. Alternatively, the set of disc carriers may be located on one side of the discs. In embodiments of the epilator where there is a plurality of sets of discs and disc carriers, the hair removal assembly may comprises alternating sets of discs and disc carriers.

The discs and disc carriers may be constructed as entirely separate elements which are then assembled to form the hair-plucking assembly. In such embodiments, the discs provide a tweezing action, and the disc carriers prevent the skin from being drawn up during tweezing.

In other embodiments, the discs and disc carriers may be constructed as a single element which has both a tweezing function and a function which prevents the skin from being lifted. In further embodiments, the discs and disc carriers are separate elements and yet work in tandem to provide tweezing and which prevent the skin from being lifted during tweezing.

The shape of the discs in the epilator is not limited, and the discs may have a substantially uniform diameter or non-uniform diameter. For example, the discs may all be round, or they may be oval or ellipsoid. The discs may all have the same shape, or they may have different shapes. The discs may optionally have cutouts or mate with another disc, or a portion of another disc, in the set. Each set of discs may also comprise discs which have differing shapes or diameters within the same set.

A spacer element may optionally be located between individual discs in a set of discs, between individual disc carriers in a set of disc carriers, or between respective sets of discs and disc carriers.

The disc carriers may be individual structural elements. For example, the disc carriers may have an overall disc-like shape. Alternatively, the disc carriers may be formed on a single unitary element which rotates during use of the device, and the discs may protrude through this unitary element.

The materials from which the discs and disc carriers are formed is not critical. Examples of such materials are plastic, metal, composite, ceramic, and combinations thereof. In one embodiment, the discs are formed from stainless steel and the disc carriers are formed from plastic.

The hair-plucking assembly and its component discs and disc carriers may be mounted on a shaft. This shaft may be curved, thereby providing an arcuate epilation surface, or the shaft may be straight, thereby providing a flat epilation surface

The discs and discs carriers may rotate at a constant rate of speed or at a variable rate of speed. Although the rotational speed is not critical, the discs and disc carriers rotate at a speed sufficient to catch hairs and pluck them from a user's skin. The discs and the discs carriers may rotate at the same rate or at different rates of speed.

The hair-plucking assembly may be removably or fixedly mounted to the epilator housing. If the hair-plucking assembly is removable from the housing, cleaning and repair or replacement of the component parts is facilitated.

The means by which the epilator motor receives power is not critical. For example, the epilator may be powered by a battery or by a domestic power line. The battery may be a conventional rechargeable or disposable battery, and may be user-replaceable or non-user replaceable.

The epilator may optionally comprise a housing cap removably mounted on the housing. The housing cap may have an opening through which the hair-plucking assembly partially extends.

In addition to the embodiments described above, the epilator may have one or more additional components which are known in the art of grooming. For example, the epilator may have a shaver assembly for cutting hair, or a vibrational assembly for providing a massaging effect. The epilator may also have a light assembly for illuminating the area of hair removal. In this manner, the user advantageously has multiple grooming tools located in a single hand-held unit. The additional components or tools may be mounted within or on the side of the housing, or on the end of the epilator opposite to the hair-plucking assembly.

### BRIEF DESCRIPTION OF THE DRAWINGS

FIG. 1A shows a front plan view of a first embodiment of the present invention.

FIG. 1B shows a side cross-sectional view of the embodiment of FIG. 1A along sight line 1B.

FIG. 1C shows a front cross-sectional view of the embodiment of FIG. 1A along sight line 1C.

FIG. 2A illustrate a perspective view of a second embodiment of the present invention.

FIG. 2B. illustrates a close-up perspective view of the hair-plucking assembly shown in FIG. 2A. The discs and disc carriers which form part of the hair-plucking assembly are mounted on a curved shaft.

FIG. 2C illustrates perspective and plan views of a disc assembly of the hair-plucking assembly of FIG. 2B.

FIG. 3 provides an expanded view of the disc assembly shown in FIG. 2C.

### DETAILED DESCRIPTION OF THE INVENTION

The present invention will now be described with reference to the Figures, wherein like numerals correspond to like elements.

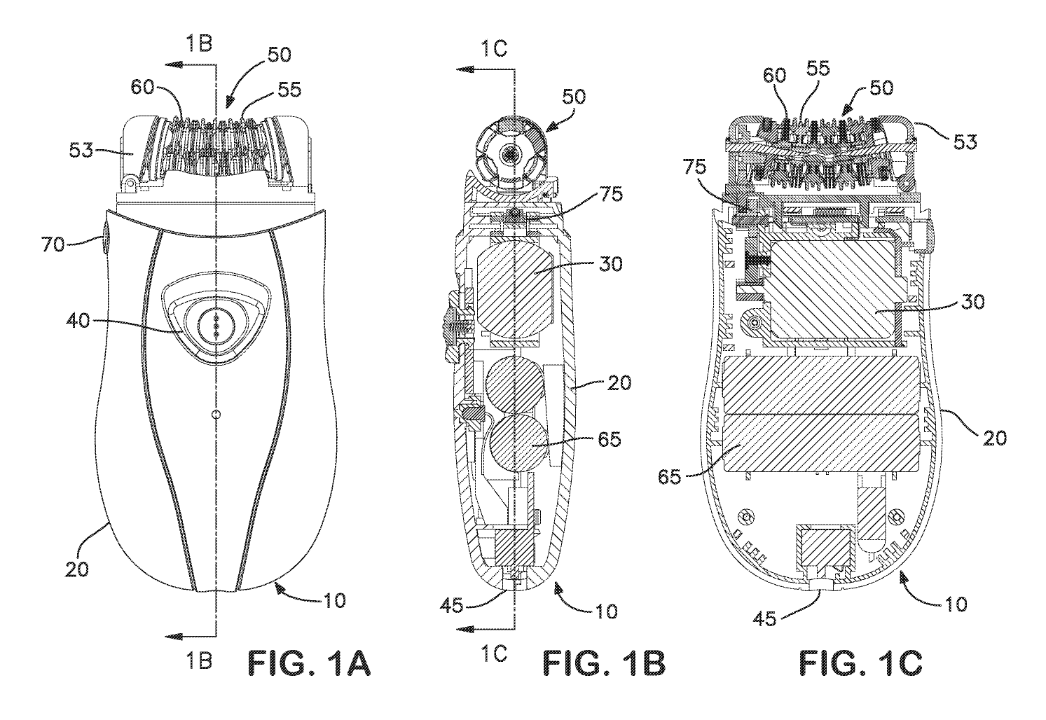

FIGS. 1A-1C illustrate a first embodiment of an epilator 10 according to the present invention. The epilator 10 comprises a housing 20, a motor 30 located within the housing, and a hair plucking assembly 50 located on at the head of the epilator. The housing 20 is ergonomically shaped to facilitate easy and comfortable gripping by a user's hand. The housing may be formed of any convenient material, such as plastic or a composite. The housing may also be coated with a substance such as a rubber to increase the tactile feel of the housing and to reduce slippage from the user's hand.

The orientation of the housing 20 in relation to the hair-plucking assembly 50 will vary depending on the particular design of the device. In general, the hair-plucking assembly will be located at one end of the housing which may be referred to as the “head” of the epilator.

The hair-plucking assembly 50 partly extends through an opening in the housing cap 53 located at the head of the epilator. In the embodiment illustrated in FIGS. 1A-1C, the housing cap 53 is removably mounted to the housing 20, thereby allowing the housing cap and its component hair-plucking assembly 50 to be readily cleaned, maintained, or replaced. The housing cap 53 can be detached from the housing 20 by pressing release button 70, and re-attached to the housing by a snap fitting or other convenient means. In other embodiments, the hair-plucking assembly or the housing cap can be fixedly mounted to the housing.

The motor 30 contained within the housing 20 can be any type of small motor which successfully drives the epilator and any associated optional peripherals. The motor 30 can be powered by a battery or by a domestic electrical power line.

In the embodiment illustrated in FIGS. 1A-1C, the epilator 10 is powered by a pair of rechargeable batteries 65. To charge the batteries 65, the user attaches a domestic power line (not illustrated) to socket 45 located on the bottom of the epilator. After the batteries are sufficiently charged, the power line is detached and the epilator is ready for use. The epilator can also be used in a corded mode, that is, while the epilator is attached to the domestic power line. In a further embodiment (not illustrated), the batteries 65 are disposable and are replaced by the user when their power is exhausted. In a further embodiment (not illustrated), the epilator is powered by the domestic power line and does not employ batteries. A power switch 40 allows the user to active or to turn off the epilator.

A gear reduction system (shown generally as 75) is located in the housing and is operationally connected to the motor 30 and the hair-plucking assembly 50. The gear reduction system 75 converts the rotational speed produced by the motor to the proper rotational speed of the hair-plucking assembly. Any suitable gear reduction system may be used in the present invention. In addition, any system that provides adequate rotational motion to the hair-plucking assembly may be incorporated into the present invention.

The hair-plucking assembly 50 extends partly through the housing cap 53 and comprises a plurality of sets of rotating discs 60 and a plurality of sets of rotating disc carriers 55. The discs and disc carriers are mounted on a curved shaft and are driven by the motor 30.

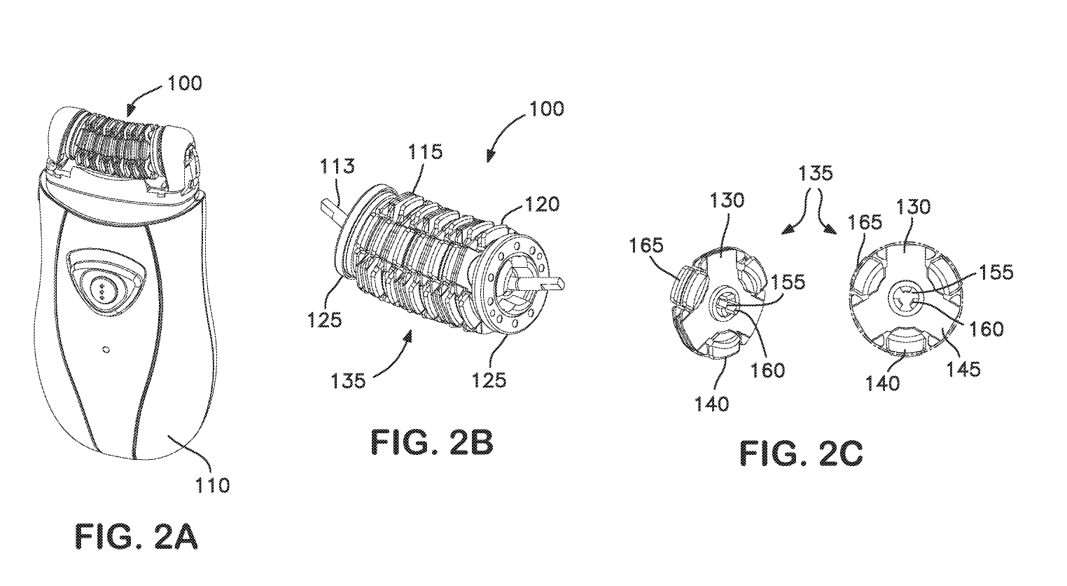

FIG. 2A illustrates a second embodiment of an epilator 110 according to the present invention. The epilator 110 has a hair-plucking assembly 100 located at its head. The hair-plucking assembly 100 is mounted on a curved shaft. The epilator may also have bearings on opposite ends of the shaft in order to provide for smooth and steady rotation while the rotational speed is transmitted from the motor. The hair-plucking assembly 100 can rotate in a clockwise or counterclockwise direction, and its different components can rotate at the same speed or at different speeds.

FIG. 2B illustrates the hair-plucking assembly 100 in greater detail. The hair-plucking assembly 100 includes a plurality of disc assemblies 135 comprising discs 115 and disc carriers 120. The discs 115 and disc carriers 120 are mounted on a curved shaft 113. The shaft 113 may have a greater or lesser degree of curvature, which will depend on the particular design. The shaft does not need to be curved, and in certain embodiments may be straight. The shaft 113 may be manufactured from any appropriate material, such as a rigid or flexible polymer or plastic.

The disc assemblies 135, discs 115, and disc carriers 120 can be prepared from any suitable material without limitation, such as metal, plastic, and composites. In one embodiment, the portions of the disc assemblies intended for contact with hair are made of a metal, and the portions of the discs assemblies which come in contact with the shaft are made of a plastic to facilitate smooth rotation. Alternatively, these components may be made of other suitable materials known in the art.

End discs 125 are placed at the outermost positions on the shaft and sandwich the disc assemblies 135, discs 115, and disc carriers 120. The end discs 125 protect the outermost discs and/or disc carriers from possible damage caused by rubbing against the housing cap.

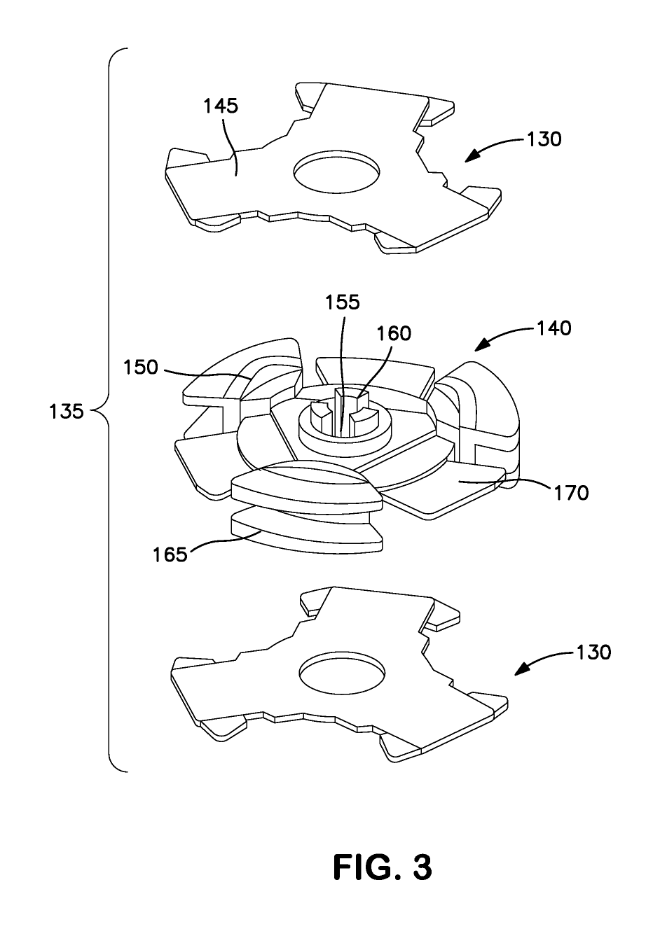

FIG. 2C illustrates perspective and plan views of a single disc assembly 135 of the hair-plucking assembly 100 of FIG. 2B. FIG. 2C illustrates that the diameter of the discs 130 is less than the diameter of the disc carriers 165. FIG. 3 illustrates an expanded view of the disc assembly 135 shown in FIG. 2C. Other disc assembly structures are possible and within the scope of the present invention.

In FIGS. 2C and 3, the discs and disc carriers are provided as disc assemblies 135 which provide a tweezing action to remove hairs from the body. Each disc assembly 135 comprises three distinct elements: an inner disc 140, and a pair of outer discs 130 located on opposite sides of the inner disc 140. The outer discs 130 have three radially extending arms 145 oriented about 120° apart. Each arm 145 terminates in a flattened peripheral portion.

There may be any number of disc assemblies 135 in accordance with the present invention. For example, there may be six disc assemblies, each of which has three sets of tweezing discs. Each disc assembly 135 is identical with respect to clockwise or counterclockwise movement, although there may be any number of identical or dissimilar disc assemblies.

The inner disc 140 also has three radially extending arms 170 which terminate in a flattened peripheral portion. The inner disc 140 may be a single disc or it may be formed by two or more separate elements joined together. In this embodiment of the invention, the inner disc 140 has three sets of disc carriers 165. The disc carriers 165 extend beyond the radius of the outer discs 130 and the peripheral arms 170 of the inner disc 140. Accordingly, the diameter of the disc carriers 165 is greater than the diameter of the outer discs 130 and the peripheral arms 170 of inner disc 140.

Although the primary function of the disc carriers 165 is to maintain the discs at a predetermined distance from the skin and to keep the skin from being lifted while hairs are being plucked, the disc carriers 165 can optionally be fitted with pinchers 150 which provide additional surfaces for removal of hairs. For example, each outer surface of the disc carrier 165 may have a pair of opposing pinchers 150. The pinchers 150 extend beyond the plane of radially-extending arms 145 such that as the disc assembly 135 rotates, pinchers 150 come into contact with and thereby impart clamping forces to the radially extending arm 145 of adjacent disc assemblies 135 during rotation. This action causes adjacent disc assemblies 135 to form traps. The disc assemblies 135 can be configured and assembled within the device such that pinchers 150 of each disc assembly remain adjacent to radially extending arms 145 of adjacent disc assemblies 135 during rotation of all disc assemblies.

When the epilator is activated, the outer discs 130 and inner disc 140 rotate together at an angle along the curved shaft 113. Flattened arm portions, e.g. 145 and 170, of adjacent inner and outer discs press against each other, thereby forming two traps to grasp hair and hold hairs. In other words, two traps are formed to grasp hairs when opposing clamping forces are applied to flattened peripheral portions of adjacent disc elements. The action of the inner and outer discs 130 and 140, or their peripheral portions 145 and 170, moving from an opened position to a closed position causes the traps to form. Six traps may be activated for a 360° rotation of the disc assembly 135.

The diameter of the disc carriers 165 can be any diameter which is greater or lesser than the diameter of the discs 130. In one embodiment, the disc carriers 165 are 0.1-3 mm greater in diameter than the discs 130. In another embodiment, the disc carriers have a diameter which is 0.6 mm greater than the diameter of the discs.

The central portion of each outer disc 130 has a hole which is sized and configured to snugly mate with the central portion of the inner disc 140. Adjacent discs and disc assemblies engaged each other by engagement means. In the embodiment illustrated in FIGS. 2B-3, these engagement means comprise engagement protrusions 160 and engagement recessions 155. The engagement protrusions 160 and recessions 155 are angularly offset and ensure that adjacent disc assemblies interact with each other to form effective hair traps as discussed above. The configurations, dimensions, and tolerances of all components are designed such that the components fit snuggly together and remain engaged during operation of the device.

In alternative embodiments of the invention, the discs and disc carriers may be constructed as entirely separate elements which are mounted on a shaft. The discs may be sets of flat or curved plates or arms which alternate with, or are interposed by, disc carriers which extend from the circumference of the discs. Other embodiments within the scope of the claims will be evident to those of skill in the art.

Further examples of disc assemblies are provided by U.S. Pat. No. 6,123,713, assigned to K.I.S. Ltd., the contents of which are incorporated herein in their entirety.

The epilator may have one or more additional optional components which are known in the art of grooming, such as a shaver assembly for trimming hairs, a vibrational assembly for a massaging effect, or a light assembly for illuminating the area of hair removal. The additional components may be mounted at any convenient location on or within the housing so as not to interfere with epilation.

While the invention has been particularly shown and described with reference to particular embodiments, those skilled in the art will understand that various changes in form and details may be made without departing from the spirit and scope of the invention.

What is claimed is:

1. A hair removal device comprising:
a housing;
a motor located in the housing; and
a hair-plucking assembly for removal of hair from a user's skin,
wherein the hair-plucking is assembly mounted to the housing and operationally connected to the motor, the hair-plucking assembly comprises at least one set of rotating discs and at least one set of rotating disc carriers, and the diameter of the discs is different than the diameter of the disc carriers.

2. The hair removal device according to claim 1, wherein the discs provide a tweezing action during use of the device.

3. The hair removal device according to claim 1, wherein the disc carriers are structurally configured to prevent a user's skin from being lifted while the discs are plucking hair from the skin.

4. The hair removal device according to claim 1, wherein the disc carriers maintain the discs at a predetermined distance from a user's skin.

5. The hair removal device according to claim 1, wherein the diameter of the disc carriers is greater than the diameter of the discs.

6. The hair removal device according to claim 1, wherein the diameter of the disc carriers is less than the diameter of the discs.

7. The hair removal device according to claim 1, wherein the diameter of the disc carriers is 0.1-3 mm greater than the diameter of the discs.

8. The hair removal device according to claim 1, wherein the hair-plucking assembly comprises a plurality of sets of discs, a plurality of sets of disc carriers, or both.

9. The hair removal device according to claim 1, wherein the set of discs comprises a pair of discs.

10. The hair removal device according to claim 1, wherein the set of discs comprises a set of two outer discs and an inner disc, wherein the outer discs have the same diameter and the inner disc has a smaller diameter than the outer discs.

11. The hair removal device according to claim 1, wherein the disc carriers are located on opposite sides of the discs.

12. The hair removal device according to claim 1, wherein the hair removal assembly comprises alternating sets of discs and disc carriers.

13. The hair removal device according to claim 1, wherein the hair-plucking assembly comprises a plurality of identical disc assemblies.

14. The hair removal device according to claim 1, wherein the discs have a uniform or non-uniform diameter.

15. The hair removal device according to claim 1, further comprising a spacer element between the discs, disc carriers, or both.

16. The hair removal device according to claim 1, wherein the disc carriers are located on a single unitary element, and the discs protrude through the unitary element.

17. The hair removal device according to claim 1, wherein the discs, the disc carriers, or both, are formed from plastic, metal, composite, ceramic, or combinations thereof.

18. The hair removal device according to claim 1, wherein the discs move from an open position to receive a hair on the user's skin and a closed position when the hair is plucked.

19. The hair removal device according to claim 1, wherein the hair-plucking assembly is mounted on a shaft.

20. The hair removal device according to claim 1, wherein the shaft is curved or straight.

21. The hair removal device according to claim 1, wherein the discs and discs carriers rotate at a constant rate of speed or at a variable rate of speed.

22. The hair removal device according to claim 1, wherein the discs and the discs carriers rotate at the same rate or at different rates of speed.

23. The hair removal device according to claim 1, wherein the hair-plucking assembly is removably or fixedly mounted to the housing.

24. The hair removal device according to claim 1, wherein the motor is powered by a battery or by a domestic power line.

25. The hair removal device according to claim 1, wherein the device further comprises a housing cap removably mounted on the housing, wherein the housing cap has an opening through which the hair-plucking assembly partially extends.

26. The hair removal device according to claim 1, wherein the device further comprises a shaver assembly, a light assembly, or a vibrational assembly, which assembly is mounted on or within the housing.
